(19) United States
(12) Patent Application Publication
(10) Pub. No.: US 2003/0088257 A1
(43) Pub. Date: May 8, 2003

(54) Directional endo-illuminator

(76) Inventors: Carl C. Awh, Brentwood, TN (US); Michael S. Poulsen, House Springs, MO (US)

Correspondence Address: THOMPSON COBURN, LLP, ONE FIRSTAR PLAZA, SUITE 3500, ST LOUIS, MO, 63101, US

(21) Appl. No.: 09/992,232

(22) Filed: Nov. 6, 2001

(51) Int. Cl. A61B 17/24

(52) U.S. Cl. 606/161

(57) ABSTRACT

A microsurgical endo-illuminator is provided with a distal end portion of an optic fiber that projects from a tubular sleeve of the illuminator and can be caused to bend relative to the illuminator sleeve by manual manipulation of a mechanism on a handle of the illuminator.

### BACKGROUND OF THE INVENTION

(1) Field of the Invention

The present invention pertains to a microsurgical illuminator used primarily in ophthalmic surgery. The illuminator has a handle and a tubular sleeve and a distal end portion of an optic fiber projecting from the sleeve can be caused to bend relative to the sleeve by manual manipulation of a mechanism on the handle.

(2) Description of the Prior Art

In ophthalmic surgery, various different types of instruments are available for use by the surgeon to transmit light to illuminate a surgical site in the interior of the eye. The typical microsurgical illuminator comprises a handle with a small cylindrical metal sleeve tip projecting from a distal end of the handle. An optic fiber, having a proximal end with a connector for coupling to a source of light, passes through the center of the handle and the sleeve of the probe. The distal end of the optic fiber is positioned adjacent the distal end of the sleeve. In instruments of this type, the sleeve can project straight from the handle of the instrument or can have a slight bend or curve as it projects from the instrument handle.

Efficient delivery of illuminating light in the eye interior toward the anterior or front portion of the retina is often awkward to the surgeon using a straight tip illuminator. This is due to the positioning of the incision or instrument entry site in the eye relative to the target area or surgical site of the light being transmitted. This is illustrated in FIG. 1 where an area A of the eye interior is inaccessible to the straight tip of the illuminator shown. The use of curved tip illuminators such as that shown in FIG. 2 allows for a greater range of coverage inside the eye, thereby overcoming the disadvantages of the straight tip illuminator probe discussed above. However, curved illuminators cannot be inserted through straight cannulas and therefore must be directed through the eye incision site itself.

The optimal deliver of illuminating light to a surgical site in the eye requires that the light be directed perpendicular to the target area of the surgical site. Directing a straight tip illuminator at anterior or forward portions of the retina causes the approach angle, or angle of incidence of the light, to be large. In this situation the optimal delivery of light to the surgical site cannot be achieved. Additionally, torquing or manipulating the tubular sleeve of the straight tip illuminator in the entry incision to reduce the angle of approach of the light to the surgical site in these awkward areas often produces excessive, and sometimes harmful stresses around the incision of the eye. Often the only way for the surgeon to overcome this situation is to create a second incision site for insertion of the illuminator.

These problems can be overcome by using a curved tip illuminator that can effectively eliminate the use of a secondary incision site since an increased area in the eye interior is accessible from the single entry site as illustrated in FIG. 2. Currently available curved tip illuminators are able to access more anterior or forward areas of the eye interior than can be achieved with straight tip illuminators. However, because their curvatures are fixed, curved tip illuminators are not efficient at directing illuminating light to areas even more anterior or more forward in the eye that would require a tighter bend or curvature of the illuminator sleeve tip, or areas at the far end or posterior of the retina which would require a straight tip illuminator due to the approach angle.

To overcome these disadvantages of prior art straight and curved microsurgical illuminators, what is needed is an adjustable directional illuminator that is capable of reducing the approach angle or angle of incidence of light toward the surgical site, thereby providing ease of access and reduced instrument manipulation at the target site, reduced tissue stress at the point of entry, and improved illumination by directing the light more perpendicular to the target surgical site.

### SUMMARY OF THE INVENTION

The directional endo-illuminator of the present invention may be constructed having either a disposable hand piece or a reusable hand piece and, although described as transmitting light, it may also be employed in providing aspiration and/or suction. The directional endo-illuminator makes use of a shape memory thermoplastic in the optic fiber of the illuminator to steer and direct the flexible optic fiber to a surgical target site. In the preferred embodiment the plastic is polymethylmethacrylate (PMMA). Whether the target site lies in the posterior or anterior portions of the eye interior, the directional endo-illuminator can easily deflect to any angle between 0° (or a straight configuration) and 90° or more. The flexible nature of the thermal setting plastic of the optic fiber allows variable adjustment of the bend angle of the fiber to deliver illuminating light to the target site. Additionally, cannulas may be used in the incision site of the eye because the endo-illuminator, when in its straight configuration, can be inserted through the cannula to position the tip of the illuminator in the interior of the eye, and then a bend can be created at the tip of the illuminator in the eye interior. The directional endo-illuminator is especially useful when accessing anterior portions of the retina, or areas that are difficult or awkward to access using traditional straight illuminators.

The directional endo-illuminator of the invention is basically comprised of a handle having an interior bore passing through its center and having a recess formed in a side of the handle communicating with the interior bore. A tubular sleeve projects from a distal end of the handle and is received in the bore for axial sliding movement relative to the handle. A finger pad positioned in the recess is connected to the sleeve and manipulating the finger pad axially through the recess causes the sleeve to be moved between a pushed forward position where it projects its greatest distance from the distal end of the handle, and a pulled back position where the sleeve projects its shortest distance from the distal end of the handle. A length of optic fiber enters the handle bore at the handle proximal end and a distal end portion of the optic fiber passes through the bore and the sleeve tip. The proximal end of the fiber is connected to a standard light source connector.

The optic fiber is constructed of PMMA plastic and a distal end portion of the optic fiber that passes through the sleeve has been heated and formed in a pre-bent 90° bend in its preferred embodiment. When the finger pad of the instrument is pushed forward, it extends the sleeve to its pushed forward position in which the distal end portion of the optic fiber is completely contained inside the tubular sleeve. When the finger pad is moved to its pulled back position, the sleeve is also moved back to its pulled back position causing the bent distal end portion of the optic fiber to be gradually exposed at the distal end of the sleeve. As the distal end portion of the optic fiber is exposed at the end of the sleeve, it gradually bends from the initial straight configuration of the sleeve toward the 90° pre-bent configuration of the fiber. In this manner, the optic fiber contained in the sleeve can be adjustably positioned through any angle between 0° when the optic fiber distal end portion is entirely contained in the tubular sleeve at its pushed forward position, to a 90° bend when the optic fiber distal end portion projects from the sleeve distal end with the sleeve moved to its pulled back position.

In a variant embodiment of the endo-illuminator, the optic fiber has an interior bore through its length that can be used for aspiration and/or suction at the surgical site.

In use of the directional endo-illuminator, the optic fiber connector is first attached to a light source. With the finger pad in its pushed forward position, the optic fiber is contained in the sleeve which projects in a straight line from the distal end of the handle. The sleeve is then inserted through a cannula positioned in an incision in the eye or inserted directly through the incision, positioning the sleeve in the eye interior. The finger pad is then slowly moved toward the rear of the handle causing the sleeve to slowly move toward its pulled back position relative to the handle. This, in turn, causes the pre-bent distal end portion of the optic fiber to gradually bend from its straight configuration toward its 90° configuration. The bending of the fiber allows optimal positioning of the fiber tip to areas where a straight fiber may not reach. Rotation of the entire instrument about its center axis may be necessary to further direct the optic fiber tip. Once the proper location of the fiber tip is achieved, illuminating light can then be delivered to the site of interest. Retraction of the fiber tip into the sleeve is performed by first pushing the finger pad forward, causing the sleeve to move toward its pushed forward position and causing straightening of the bent portion of the optic fiber projecting from the sleeve. With the optic fiber contained in the sleeve, the sleeve is then pulled back through the surgical entry site.

### DESCRIPTION OF THE DRAWINGS

Further objects and features of the present invention will be revealed in the following detailed description of the preferred embodiment of the invention and in the drawing figures, further comprising:

FIG. 1 is an illustration of a conventional straight microsurgical illuminator employed in ophthalmic surgery;

FIG. 2 is a view similar to FIG. 1 but showing a conventional curved microsurgical illuminator;

FIG. 3 is a partially sectioned view of the directional endo-illuminator of the invention with the curved distal end portion of the optic fiber projecting from the sleeve;

FIG. 4 is a partial sectioned view of the instrument sleeve with the optic fiber in its straight configuration;

FIG. 5 is a partial sectioned view showing the connections of the sleeve and optic fiber in the instrument handle;

FIG. 6 is a partial sectioned view showing the detail of the optic fiber in its curved configuration; and

FIG. 7 is an end view of the instrument handle showing the optic fiber in its curved configuration.

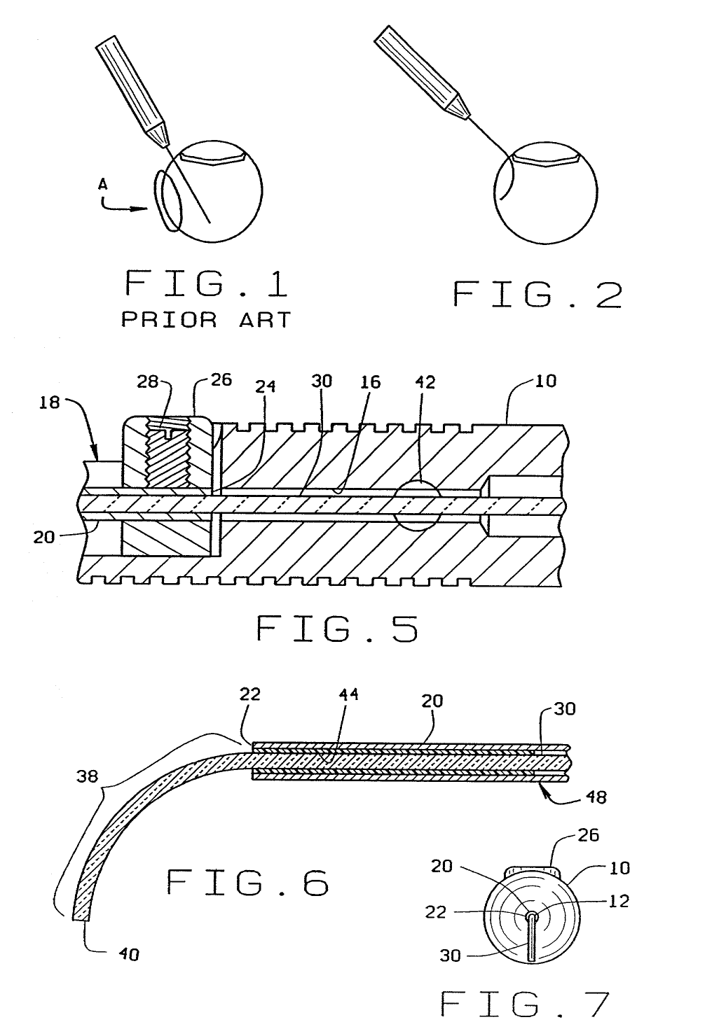

### DETAILED DESCRIPTION OF THE PREFERRED EMBODIMENT

The directional endo-illuminator of the invention is described herein as transmitting illuminating light for use in microsurgery of the eye. However, the probe is equally well suited for use in providing aspiration and/or suction. In addition, the probe can be designed as a disposable instrument or as a reusable instrument.

The directional endo-illuminator is provided with an elongated narrow handle or hand piece 10 having opposite distal 12 and proximal 14 ends. The handle 10 is dimensioned to a size similar to that of a pencil to fit comfortably in the surgeon's hand. The handle is preferably manufactured of a disposable medical grade plastic. A hollow bore 16 extends through the center of the handle from its distal end 12 to its proximal end 14. The bore 16 enlarges slightly adjacent the proximal end 14 of the handle. A recess 18 is formed into a side of the handle and intersects the center bore 16. The recess 18 extends axially along a short length of the handle forming an axial slot.

A cylindrical narrow tube or sleeve 20 of stainless steel is received in the bore 16 at the distal end 12 of the handle for sliding movement of the sleeve 20 through the bore. The sleeve 20 projects from the handle distal end 12 to a distal end 22 of the sleeve. The opposite proximal end 24 of the sleeve is positioned in the recess or slot 18 of the handle.

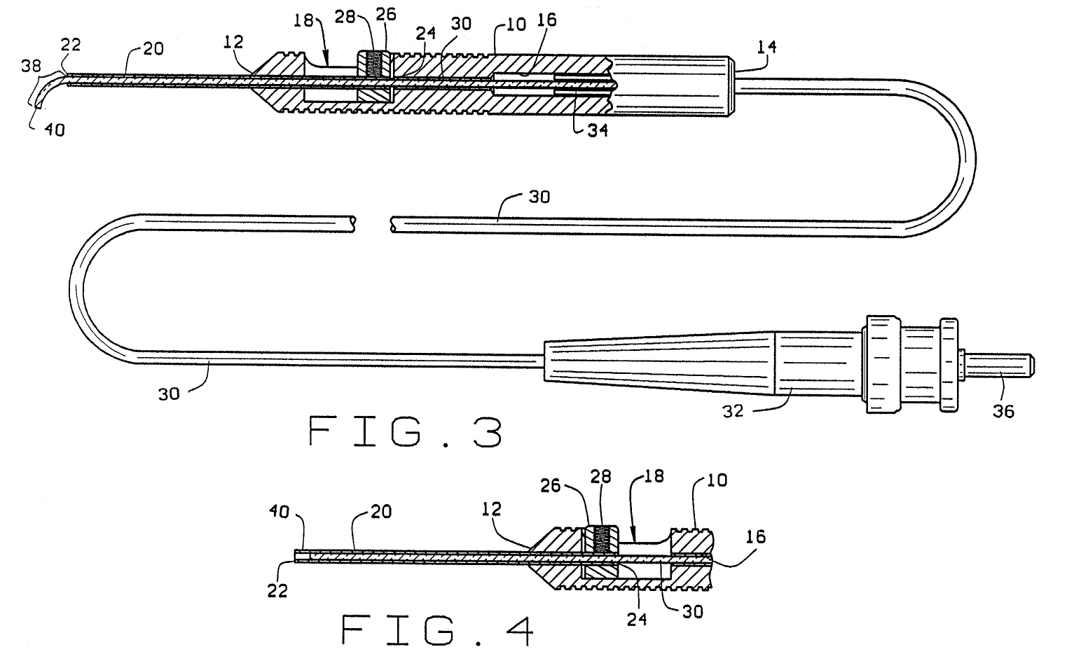

A finger pad 26 is positioned in the slot 18 for axial sliding movement of the finger pad through the slot between a pushed forward position of the finger pad 26 shown in FIG. 4 and a pulled back position of the finger pad shown in FIG. 3. The finger pad 26 is preferably constructed of disposable medical grade plastic. The finger pad has a hole into which the sleeve proximal end 24 is inserted. A set screw 28 secures the finger pad 26 to the proximal end 24 of the sleeve. Thus, moving the finger pad 26 to its pushed forward position shown in FIG. 4 will also move the sleeve 20 through the handle bore 16 to its forward most position or pushed forward position relative to the handle 10 where it projects its greatest distance from the handle distal end 12. Moving the finger pad 26 to its pulled back position shown in FIG. 3 will also move the sleeve 20 to its pulled back position relative to the handle 10 where the sleeve distal end 22 projects its shortest distance from the handle distal end 12. In the preferred embodiment of the invention, the travel distance of the finger pad 26 in the slot 18 and of the sleeve distal end 22 is 25 mm.

A length of optic fiber 30 extends between the handle 10 and a connector 32. The length of optic fiber 30 between the handle 10 and the connector 32 is protected by a layer of cladding as is conventional. The proximal end 34 of the optic fiber enters the connector 32 and its cladding is removed. The exposed portion of the optic fiber extends entirely through the connector 32 and its proximal end is positioned adjacent the end of the ferrule 36 projecting from the connector 32 as is conventional in optic fiber microsurgical instruments. The distal end of the optic fiber 30 enters the handle center bore 16 at the handle proximal end 14. Inside the handle center bore 16 the protective cladding of the optic fiber is removed and the optic fiber enters the handle recess 18 and the sleeve proximal end 24 in the recess. The exposed optic fiber extends to a distal end face 40 of the fiber positioned just inside the sleeve distal end 22 when the sleeve is moved to its forwardmost position shown in FIG. 4. The intermediate portion of the optic fiber extending through the handle center bore 16 just behind the handle recess 18 is secured stationary relative to the handle 10 by a set screw 42 that passes through the side of the handle and engages against the exterior of the optic fiber as show in FIG. 5. With the optic fiber distal end face 40 positioned just inside the distal end 22 of the sleeve when the sleeve is moved to its pushed forward position, the distal end portion 38 of the fiber projects from the sleeve distal end 22 when the finger pad 26 and the sleeve 20 are moved to their pulled back positions shown in FIG. 3. The distal end portion 38 of the optic fiber 30 that projects from the sleeve distal end 22 is shown in FIG. 6. In the preferred embodiment, the optic fiber 30 is constructed of the thermoplastic polymethylmethacrylate (PMMA). The distal end portion 38 of the optic fiber is prebent as shown in FIG. 6 so that it will curve through an angle of 90° relative to the straight sleeve 20 when the sleeve is moved completely to its pulled back position as shown in FIG. 6. Other pre-bent angles of the optic fiber distal end portion 38 greater than 90° or less than 90° may also be used. To obtain the 90° bend, the distal end portion 38 of the optic fiber 30 is bent in the configuration shown in FIG. 6 and is then heated. Thus, when the finger pad 26 of the illuminator is pushed forward, it extends the sleeve 20 to its pushed forward position in which the distal end portion 38 of the optic fiber 30 is completely contained inside the sleeve and is held in the straight configuration of the sleeve. When the finger pad 26 is moved to its pulled back position, the sleeve 20 is also moved back to its pulled back position causing the bent distal end portion 38 of the optic fiber 30 contained therein to be gradually exposed at the distal end of the sleeve. As the distal end portion 38 of the optic fiber is exposed at the end of the sleeve, it gradually bends from the initial straight configuration of the sleeve toward the 90° pre-bent configuration of the fiber distal end portion 38. In this manner, the optic fiber distal end portion 38 can be adjustably positioned through any angle between 0° when the fiber distal end portion 38 is entirely contained in the tubular sleeve 20 at its pushed forward position to a 90° bend when the fiber distal end portion 38 projects from the sleeve distal end 22 with the sleeve 20 moved to its pulled back position.

To assist the sliding of the sleeve 20 over the optic fiber distal end portion 38, the interior of the sleeve is coated with a layer 44 of a sliding material such as Teflon®. The sliding material layer 44 extends only a short distance in the sleeve interior adjacent the sleeve distal end 22. The remainder of the sleeve interior is dimensioned slightly larger than the exterior diameter of the optic fiber distal end portion 38 with its cladding removed providing an air gap between the fiber distal end portion exterior surface and the sleeve interior surface. The layer of sliding material and the air gap both reduce actuation drag and enhance the ease of sliding the sleeve 20 over the optic fiber distal end portion 38.

In a variant embodiment of the endo-illuminator, the optic fiber 30 has an interior bore 46 in FIG. 3. The hollow interior bore 46 of the optic fiber can be used for aspiration and/or suction at the surgical site. The source of aspiration and/or suction would be connected to the optic fiber proximal end 34 in addition to the connection with the light source using conventional connectors.

In use of the endo-illuminator, the optic fiber connector 32 is first attached to a light source. With the finger pad 26 in its pushed forward position, the optic fiber is contained in the sleeve 20 which projects in a straight line from the distal end of the handle. The sleeve 20 is then inserted through a cannula positioned in an incision in the eye or inserted directly through the incision positioning the sleeve in the eye interior. The finger pad 26 is then slowly moved toward the rear of the handle causing the sleeve 20 to slowly move toward its pulled back position relative to the handle. This, in turn, causes the pre-bent distal end portion 38 of the optic fiber to gradually extend from the sleeve and bend from its straight configuration toward its 90° configuration. The bending of the fiber allows optimal positioning of the fiber distal end surface 40 to areas where a straight fiber may not reach. Rotation of the entire instrument about its center axis may be necessary to further direct the optic fiber end surface 40. Once the proper location of the fiber tip is achieved, illuminating light can then be delivered to the site of interest. Retraction of the tip is performed by first pushing the finger pad 26 forward causing the sleeve 20 to move toward its pushed forward position and causing straightening of the bent distal end portion 38 of the optic fiber projecting from the sleeve. With the optic fiber contained in the sleeve, the sleeve is then pulled back through the surgical entry site.

In alternate embodiments of the invention, the sleeve 20 could be mounted stationary relative to the handle 10 and the pre-bent portion of the optic fiber could be made moveable relative to the sleeve and handle to adjust the bend in the fiber. Also, the sleeve 20 could be provided with a hole 48 for injection or suction of fluids through the hole and the air gap created between the optic fiber exterior surface and the sleeve interior surface behind the Teflon® layer 44. Furthermore, the actuation mechanism provided by the finger pad 26 can be replaced with other types of mechanisms that would produce the same axial movement of the sleeve 20, for example by a trigger mechanism manipulated by the surgeon's finger or by a squeeze mechanism that is squeezed by the surgeon's hand. In addition, a fiducial mark could be provided on the sleeve adjacent its distal end 22 to indicate to the surgeon which direction the distal end portion 38 of the optic fiber will bend as it is extended out of the distal end 22 of the sleeve. This would be useful to the surgeon in accurately positioning the sleeve in the interior of the eye before the bending movement of the fiber is commenced.

The above-described directional endo-illuminator is intended for use as a disposable instrument. If the illuminator is to be reusable, the only difference in the construction is in the dimension of the optic fiber that passes through the instrument. To accommodate various light fiber sizes, the sleeve 20 could be increased to a larger diameter.

While the present invention has been described by reference to a specific embodiment, it should be understood that modifications and variations of the invention may be constructed without departing from the scope of the invention defined in the following claims.

What is claimed:

1. A surgical instrument comprising:
an instrument handle;
a tubular sleeve projecting from the instrument handle;
a plastic optic fiber extending through the handle and the sleeve to a distal end portion that projects from the sleeve, the distal end portion of the optic fiber having an adjustable bend therein.

2. The instrument of claim 1, further comprising::
the distal end portion of the optic fiber is retractable into the sleeve.

3. The instrument of claim 1, further comprising:
the sleeve projects straight from the handle and the distal end portion of the optic fiber bends relative to the sleeve as it projects from the sleeve.

4. The instrument of claim 1, further comprising:
the sleeve is a rigid tube that projects from the handle and the distal end portion of the optic fiber has a preformed bend that is straightened when the distal end portion is retracted into the tube of the sleeve and bends when the distal end portion is extended from the tube of the sleeve.

5. The instrument of claim 1, further comprising:
the handle has a mechanism that is connected to the sleeve and selectively moves the sleeve between pushed forward and pulled back positions of the sleeve relative to the handle.

6. The instrument of claim 5, further comprising:
the optic fiber is held stationary relative to the handle and in the pushed forward position of the sleeve the distal end portion of the optic fiber is entirely contained in the sleeve and in the pulled back position of the sleeve the distal end portion of the optic fiber projects from the sleeve.

7. The instrument of claim 1, further comprising:
a hollow interior bore extends through the optic fiber.

8. The instrument of claim 1, further comprising:
the distal end portion of the optic fiber is formed of a thermoplastic and is pre-bent at an angle.

9. The instrument of claim 1, further comprising:
the optic fiber is the only optic fiber that passes through the handle and the sleeve.

10. The instrument of claim 1, further comprising:
the instrument is an illuminating probe.

11. The instrument of claim 1, further comprising:
the plastic is polymethylmethacrylate.

12. The instrument of claim 1, further comprising:
the tubular sleeve has an interior surface and there is an air gap between the sleeve interior surface and the optic fiber in the sleeve.

13. The instrument of claim 1, further comprising:
the tubular sleeve has an interior surface and a layer of sliding material between the sleeve interior surface and the optic fiber in the sleeve.

14. The instrument of claim 13, further comprising:
the layer of sliding material is located in only a portion of the sleeve creating an air gap between the sleeve interior surface and the optic fiber where the layer of sliding materiel is not located.

15. A surgical instrument comprising:
an instrument handle;
a tubular sleeve projecting from the handle;
a plastic optic fiber extending through the handle and the sleeve to a distal end portion of the fiber; and
a mechanism on the handle and connected to the sleeve to selectively move the sleeve between a pushed forward position of the sleeve where the sleeve projects a first distance from the handle and a pulled back position of the sleeve where the sleeve projects a second distance from the handle that is less than the first distance.

16. The instrument of claim 15, further comprising:
the optic fiber is secured stationary to the handle and a distal end portion of the optic fiber projects from the sleeve when the sleeve is moved to the pulled back position and the distal end portion of the optic fiber is entirely contained in the sleeve when the sleeve is moved to the pushed forward position.

17. The instrument of claim 16, further comprising:
the distal end portion of the fiber has a bend formed therein.

18. The instrument of claim 15, further comprising:
a hollow interior bore extends through the optic fiber.

19. The instrument of claim 15, further comprising:
the distal end portion of the optic fiber is formed of a thermoplastic and is pre-bent at an angle.

20. The instrument of claim 15, further comprising:
the sleeve is a rigid tube that projects from the handle and the distal end portion of the optic fiber has a preformed bend that is straightened when the distal end portion is retracted into the tube of the sleeve and bends when the distal end portion is extended from the tube of the sleeve.

21. The instrument of claim 15, further comprising:
the sleeve projects straight from the handle and the distal end portion of the optic fiber bends relative to the sleeve as it projects from the sleeve.

22. The instrument of claim 15, further comprising:
the optic fiber is the only optic fiber that passes through the handle and the sleeve.

23. The instrument of claim 15, further comprising:
the plastic is polymethylmethacrylate.

24. The instrument of claim 15, further comprising:
the tubular sleeve has an interior surface and there is an air gap between the sleeve interior surface and the optic fiber in the sleeve.

25. The instrument of claim 15, further comprising:
the tubular sleeve has an interior surface and a layer of sliding material between the sleeve interior surface and the optic fiber in the sleeve.

26. The instrument of claim 25, further comprising:
the layer of sliding material is located in only a portion of the sleeve creating an air gap between the sleeve interior surface and the optic fiber where the layer of sliding material is not located.
